(19) United States
(12) Patent Application Publication
(10) Pub. No.: US 2015/0173935 A1
(43) Pub. Date: Jun. 25, 2015

(54) ORAL ORTHOSES AND ASSOCIATED DEVICES, SYSTEMS, AND METHODS

(71) Applicant: University of Washington through its Center for Commercialization, Seattle, WA (US)

(72) Inventors: Mark S. Cooper, Seattle, WA (US); Anthony Sims, Woodstock, MD (US)

(21) Appl. No.: 14/416,249

(22) PCT Filed: Mar. 13, 2013
(86) PCT No.: PCT/US13/30909

§ 371 Date: Jan. 21, 2015

Related U.S. Application Data
(60) Provisional application No. 61/674,803, filed on Jul. 23, 2012.

(51) Int. Cl. A61F 5/37; A61C 5/14

(52) U.S. Cl. CPC A61F 5/37; A61C 5/14

(57) ABSTRACT

Orthoses, screening kits, and associated devices, systems, and methods are disclosed herein. An orthosis (100) configured in accordance with a particular embodiment includes a left spacer (102) configured to be clenched between left lateral posterior teeth, a right spacer (104) configured to be clenched between right lateral posterior teeth, and a connector (106) extending therebetween. The connector is flexible to accommodate differences in relative positioning of left and right lateral posterior teeth in different patients. The orthosis, when operably positioned within an oral cavity of a patient, is configured to cause asymmetrical spacing between upper and lower posterior teeth relative to a sagittal plane. A screening kit configured in accordance with a particular embodiment includes sets of orthoses individually configured to cause different levels of asymmetrical or at least generally symmetrical spacing between upper and lower posterior teeth relative to the sagittal plane.

### CROSS-REFERENCE TO RELATED APPLICATION

This application claims priority to pending U.S. Provisional Application No. 61/674,803, filed Jul. 23, 2012, which is incorporated herein by reference in its entirety.

### TECHNICAL FIELD

The present technology relates generally to oral orthoses (e.g., oral orthoses used to screen for and/or to implement treatments for neurological, neuropsychiatric, and/or other disorders).

### BACKGROUND

Oral biomechanical input (e.g., movement of, positioning of, and/or force on anatomical structures via the oral cavity) can have wide-ranging physiological effects. In some cases, oral biomechanical input has been shown to be useful for the treatment of disease. For example, oral biomechanical input has been shown to temporarily reduce symptoms in at least some patients diagnosed with movement disorders (e.g., focal dystonias, Tourette's syndrome, etc.). The etiologies of movement disorders are often complex and their clinical presentations can vary significantly from patient to patient. It is theorized that some movement disorders may be associated with misalignment of the temporomandibular joints. In patients diagnosed with one or more of these movement disorders, oral orthoses can be used to at least temporarily improve the alignment of the temporomandibular joints. This form of treatment has been shown to substantially relieve symptoms, including debilitating symptoms, of at least some of these movement disorders with few or no undesirable side effects. Oral orthoses also have significant potential for the treatment of other disorders (e.g., other neurological and/or neuropsychiatric disorders). Currently, however, oral orthoses remain underutilized as a treatment option. For this and/or other reasons, there is a need for further innovation in this field.

### BRIEF DESCRIPTION OF THE DRAWINGS

Many aspects of the present technology can be better understood with reference to the following drawings. The relative dimensions in the drawings may be to scale with respect to some embodiments. With respect to other embodiments, the drawings may not be to scale. For ease of reference, throughout this disclosure identical reference numbers may be used to identify identical or at least generally similar or analogous components or features.

FIGS. 1A-1E are perspective, plan, inverted plan, front elevation, and side elevation views, respectively, illustrating a first orthosis configured in accordance with an embodiment of the present technology.

FIGS. 2A-2E are perspective, plan, inverted plan, front elevation, and side elevation views, respectively, illustrating a second orthosis configured in accordance with an embodiment of the present technology.

FIGS. 3A-3E are perspective, plan, inverted plan, front elevation, and side elevation views, respectively, illustrating a third orthosis configured in accordance with an embodiment of the present technology.

FIGS. 4A-4E are perspective, plan, inverted plan, front elevation, and side elevation views, respectively, illustrating a fourth orthosis configured in accordance with an embodiment of the present technology.

FIG. 5 is a perspective view of a screening kit configured in accordance with an embodiment of the present technology.

FIG. 6 is a plan view of a screening kit configured in accordance with another embodiment of the present technology.

### DETAILED DESCRIPTION

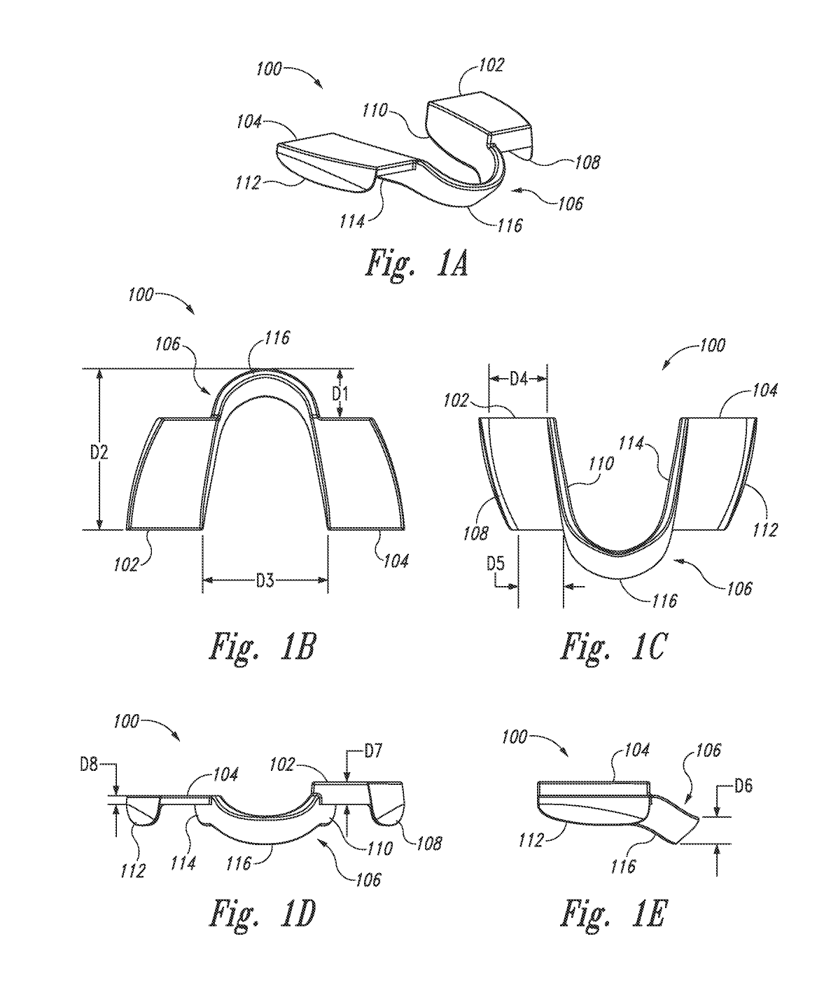

Specific details of several embodiments of the present technology are described herein with reference to FIGS. 1A-6. Although many of the embodiments are described herein with respect to selecting oral orthoses for patients diagnosed with movement disorders, other applications and other embodiments in addition to those described herein are within the scope of the present technology. For example, some embodiments may be useful for selecting oral orthoses for patients diagnosed with other disorders responsive to oral biomechanical input. Additionally, some embodiments may be useful outside the context of selecting oral orthoses. For example, in some cases, oral orthoses disclosed in the context of screening can be used for implementing treatments. It should also be noted that embodiments of the present technology can have different configurations, components, and/or procedures than those shown and described herein. Moreover, a person of ordinary skill in the art will understand that embodiments of the present technology can have components and/or procedures in addition to those shown or described herein and that these and other embodiments can be without several of the components and/or procedures shown or described herein without deviating from the present technology. The headings provided herein are for convenience only.

One possible reason for the underutilization of oral orthoses for treatment may be the technical challenge of identifying efficacious oral orthoses for different patients. Among patients having disorders responsive to oral biomechanical input, the relative efficacy of different types and degrees of oral biomechanical input typically varies from patient to patient. In a conventional screening method for selecting an oral orthosis, a clinician first performs a series of brief tests on a patient and, based on the results of the tests, the clinician selects an oral orthosis that is thought by the clinician to be well suited for treating the patient's symptom(s). Unfortunately, this conventional screening method tends to be unreliable, often leading clinicians to select suboptimal oral orthoses. Furthermore, since dental anatomies vary widely within the population, conventional oral orthoses are typically custom made for each patient to facilitate long-term comfort and durability. Due to this customization, the cost of conventional oral orthoses is often relatively high. When an unreliable screening method leads to the selection of a suboptimal oral orthosis for a given patient, making a new oral orthosis for the patient may be impractical. The patient is also unlikely to realize that an oral orthosis in use is suboptimal. Furthermore, due to the placebo effect and/or other factors, unreliable screening methods can lead clinicians to prescribe oral orthoses for patients who do not actually benefit from oral biomechanical input, leading to significant unnecessary and unrecoverable costs.

One example of a conventional screening method includes temporarily placing a screening implement (e.g., a stack of tongue depressors) at different positions within a patient's oral cavity and instructing the patient to bite down on the implement at each position. The clinician then observes any corresponding physiological effect at each position. In some cases, the clinician relies on the patient's perception and communication of these physiological effects. The position corresponding to the most beneficial perceived physiological effect is then used as a basis for the configuration of the patient's permanent oral orthosis. When performed by a skilled clinician, this conventional screening method is often at least moderately useful for identifying an efficacious type of oral biomechanical input (e.g., unilateral left vertical distraction, unilateral right vertical distraction, or bilateral vertical distraction) for a given patient, but it tends to be imprecise and/or inaccurate for identifying a degree of input that enhances (e.g., optimizes) the desired effect of the input on the patient's symptom(s). Furthermore, this conventional screening method often fails to identify potentially synergistic combinations of two or more different types of oral biomechanical input (e.g., unilateral left vertical distraction in combination with tongue restriction, and unilateral right vertical distraction in combination with tongue restriction, among others).

Instead of or in addition to differences in the efficacy of oral orthoses that apply different types of oral biomechanical input, there can be differences in the efficacy of oral orthoses that apply different degrees of the same type of oral biomechanical input (e.g., the degree to which an oral orthosis moves, positions, and/or exerts force against anatomical structures via the oral cavity). For example, in a patient having misaligned temporomandibular joints, there can be a window of mechanical adjustment that causes the patient's temporomandibular joints to move into alignment and thereby significantly reduces the patient's symptoms. In some cases, mechanical adjustment to a degree less than and/or greater than this window reduces or even eliminates the adjustment's beneficial effect. The ability of conventional screening methods to identify efficacious degrees of oral biomechanical input is typically limited by one or more variables, such as the temporary level of occlusal force exerted by the patient during the testing relative to a suitable protracted occlusal force for the patient, the patient's physiological state during the testing relative to the patient's more typical physiological state during a desired time of treatment, and a degree of correspondence between the oral biomechanical input caused by the screening implement during the screening and the oral biomechanical input that would be caused by a permanent oral orthosis. Moreover, in some cases, it may be difficult for clinicians and/or patients to accurately identify subtle physiological effects during conventional screening.

Screening devices and methods in accordance with at least some embodiments of the present technology can enhance the efficacy and/or reduce the costs associated with the therapeutic use of oral orthoses. Thus, these screening devices and methods may facilitate more reliable and/or practical selection of oral orthoses relative to conventional screening devices and methods. A screening method in accordance with a particular embodiment includes providing a patient with a set of screening orthoses having different configurations. The patient can be instructed to test some or all of the screening orthoses (e.g., in a specified sequence). The individual screening orthoses can cause oral biomechanical input that corresponds relatively closely to oral biomechanical input that would be caused by a more permanent oral orthosis having a corresponding configuration. In some cases, the individual screening orthoses are tested over a relatively long period (e.g., a day or more). For this and/or other reasons, screening methods in accordance with at least some embodiments of the present technology may reduce or eliminate inaccuracies associated with one or more of the variables described above. In some cases, the screening orthoses can be relatively low-cost and/or disposable. Furthermore, the screening orthoses can have one or more features (e.g., flexibility) that allow them to be used in patients having different dental anatomies (e.g., different bite curvatures).

Oral orthoses configured in accordance with at least some embodiments of the present technology can be used to biomechanically modulate neuronal activity (e.g., in the second (maxillary) and/or third (mandibular) branch of the trigeminal nerve). By way of theory, and not to limit the present disclosure, oral orthoses in accordance with at least some embodiments of the present technology may remove or otherwise change mechanical pressure on terminals (e.g., irritated terminals) of the auriculotemporal nerve, stretch tendons around the temporomandibular joint, stretch muscles around the temporomandibular joint, stretch ligaments around the temporomandibular joint, stimulate afferent activity to the brainstem, and/or have one or more other effects that can eliminate or reduce the severity of one or more symptoms of a disorder (e.g., one or more symptoms of a movement disorder).

In some cases, the antagonism of movement disorders by sensory stimuli can be referred to as a “sensory trick” or “geste antagoniste.” When sensory tricks are substantial, they may overcome the pathological effects of triggering events, resulting in partial or complete normalization of neurological function. In this way and/or by another suitable mechanism, oral orthoses configured in accordance with at least some embodiments of the present technology can be used to eliminate and/or reduce the severity of movement disorder symptoms. Screening kits including multiple oral orthoses configured in accordance with at least some embodiments of the present technology can be used to test different types of oral biomechanical input to develop therapeutically effective treatments on a case-by-case basis. For example, in a particular embodiment, a screening kit can include a control, oral orthoses configured to test one or more levels of bilateral vertical distraction, oral orthoses configured to test one or more levels of unilateral right-side vertical distraction, oral orthoses configured to test one or more levels of unilateral left-side vertical distraction, and/or one or more oral orthoses configured to test tongue restriction.

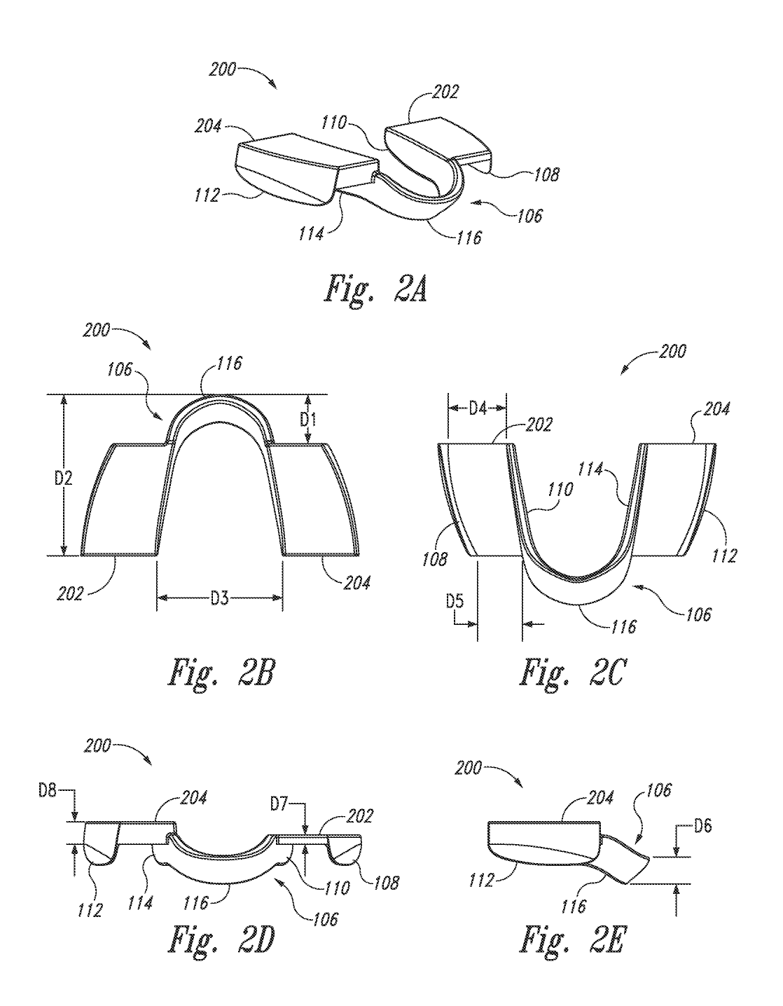

FIGS. 1A-1E are perspective, plan, inverted plan, front elevation, and side elevation views, respectively, illustrating a first orthosis 100 configured in accordance with an embodiment of the present technology. In FIGS. 1A-1E and elsewhere in FIGS. 2A-6, patient anatomy is not shown for simplicity of illustration. The first orthosis 100 can include pad-shaped left and right spacers 102, 104 and a curved connector 106 extending therebetween. When the first orthosis 100 is operably positioned within a patient's oral cavity, the left and right spacers 102, 104 can be posterior to and out of contact with the patient's anterior teeth. The first orthosis 100 can further include a left buccal flange 108 that extends downward from a buccal side of the left spacer 102, and a left lingual flange 110 that extends downward from a lingual side of the left spacer 102 when the first orthosis 100 is operably positioned within the patient's oral cavity. Similarly, the first orthosis 100 can include a right buccal flange 112 that extends downward from a buccal side of the right spacer 104, and a right lingual flange 114 that extends downward from a lingual side of the right spacer 104 when the first orthosis 100 is operably positioned within the patient's oral cavity. The connector 106 can include a flexible strap 116 extending between the left and right spacers 102, 104. For example, the flexible strap 116 can have a first end directly coupled to the left lingual flange 110 and a second, opposite end directly coupled to the right lingual flange 114. The flexible strap 116 can be configured to have anterior and posterior major sides and superior and inferior edges when the first orthosis 100 is operably positioned within the patient's oral cavity. In some embodiments, the left buccal and lingual flanges 108, 110 cause the first spacer 102 to be at least partially self centering on the patient's bottom left posterior teeth and the right buccal and lingual flanges 112, 114 cause the second spacer 104 to be at least partially self centering and the patient's bottom right posterior teeth.

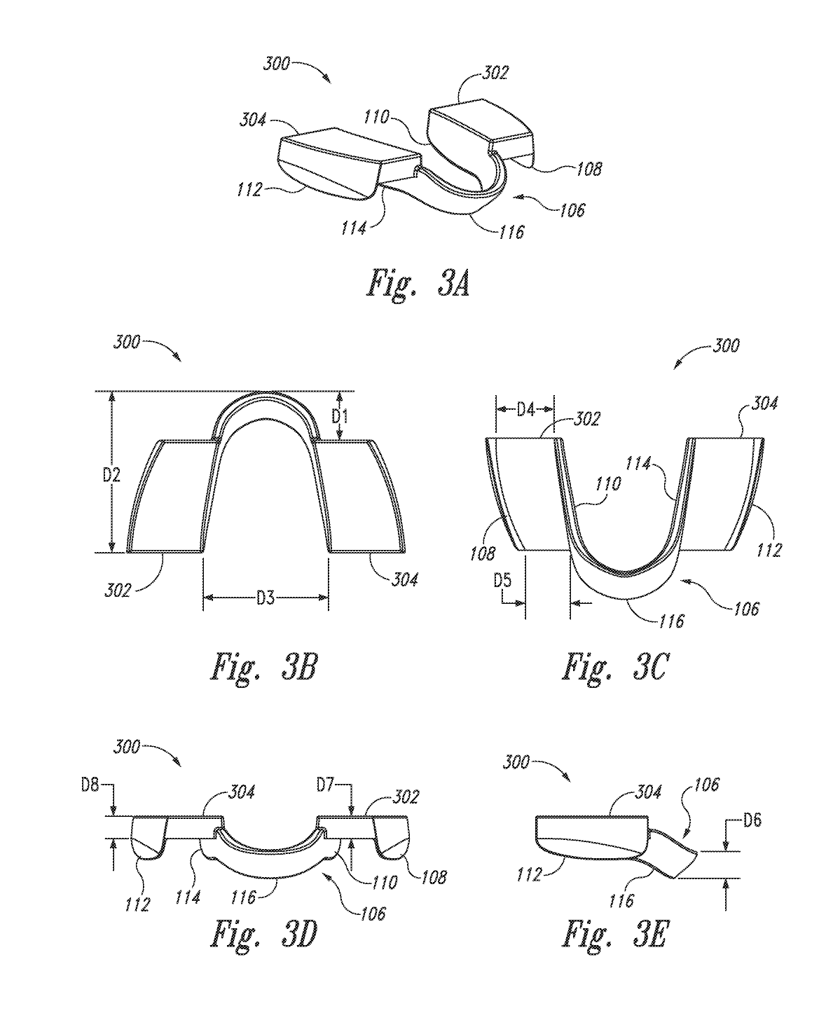

FIGS. 2A-2E are perspective, plan, inverted plan, front elevation, and side elevation views, respectively, illustrating a second orthosis 200 configured in accordance with an embodiment of the present technology. The second orthosis 200 can include pad-shaped left and right spacers 202, 204 and the curved connector 106 extending therebetween. FIGS. 3A-3E are perspective, plan, inverted plan, front elevation, and side elevation views, respectively, illustrating a third orthosis 300 configured in accordance with an embodiment of the present technology. The third orthosis 300 can include pad-shaped left and right spacers 302, 304 and the curved connector 106 extending therebetween. With reference to FIGS. 1A-3E together, the first, second, and third orthoses 100, 200, 300 can be configured to cause different spacings between the patient's upper and lower posterior teeth. For example, when the individual first, second, and third orthoses 100, 200, 300 are operably positioned within the patient's oral cavity, the left spacers 102, 202, 302 can be clenched between the patient's upper and lower left lateral posterior teeth, and the right spacers 104, 204, 304 can be clenched between the patient's upper and lower right lateral posterior teeth. In this way, the left spacers 102, 202, 302 can cause a left spacing (e.g., vertical distraction) between the patient's upper and lower left lateral posterior teeth and the right spacers 104, 204, 304 can cause a right spacing (e.g., vertical distraction) between the patient's upper and lower right lateral posterior teeth.

In some embodiments, relative to the patient's sagittal plane, the first orthosis 100 is configured to cause an asymmetrical spacing in which the left spacing is greater than the right spacing (e.g., by a value within a range from about 5 millimeters (mm) to about 15 mm), the second orthosis 200 is configured to cause an asymmetrical spacing in which the right spacing is greater than the left spacing (e.g., by a value within a range from about 5 mm to about 15 mm), and the third orthosis 300 is configured to cause an at least generally symmetrical spacing in which the left and right spacings are at least generally the same. In other embodiments, the first, second, and third orthoses 100, 200, 300 can be configured to cause other suitable spacings. For example, the first and second orthoses 100, 200 can be configured to cause different asymmetrical spacings in which the left spacing is greater than the right spacing by different degrees or different asymmetrical spacings in which the right spacing is greater than the left spacing by different degrees.

The connector 106 can be configured to accommodate anatomical differences in relative positioning of left and right lateral posterior teeth in different patients. For example, in some embodiments, at least a portion of the connector 106 is sufficiently flexible to allow the curvature of the connector 106 to change to accommodate different dental anatomies (e.g., different bite curvatures) in different patients. In other embodiments, the connector 106 can be partially or entirely rigid and bendable (e.g., at a hinge (not shown)), extendable (e.g., at an extension clasp (not shown)), or otherwise adjustable to accommodate different dental anatomies. The superior edge of the flexible strap 116 can angle downward relative to the patient's occlusal plane as the superior edge extends mesially from the left and right spacers 102, 104. Angling the superior edge downward relative to the patient's occlusal plane can reduce the degree to which the first, second, and third orthoses 100, 200, 300 encroach upon the patient's resting tongue position. As discussed below, however, in some cases, encroaching upon the patient's resting tongue position may be desirable.

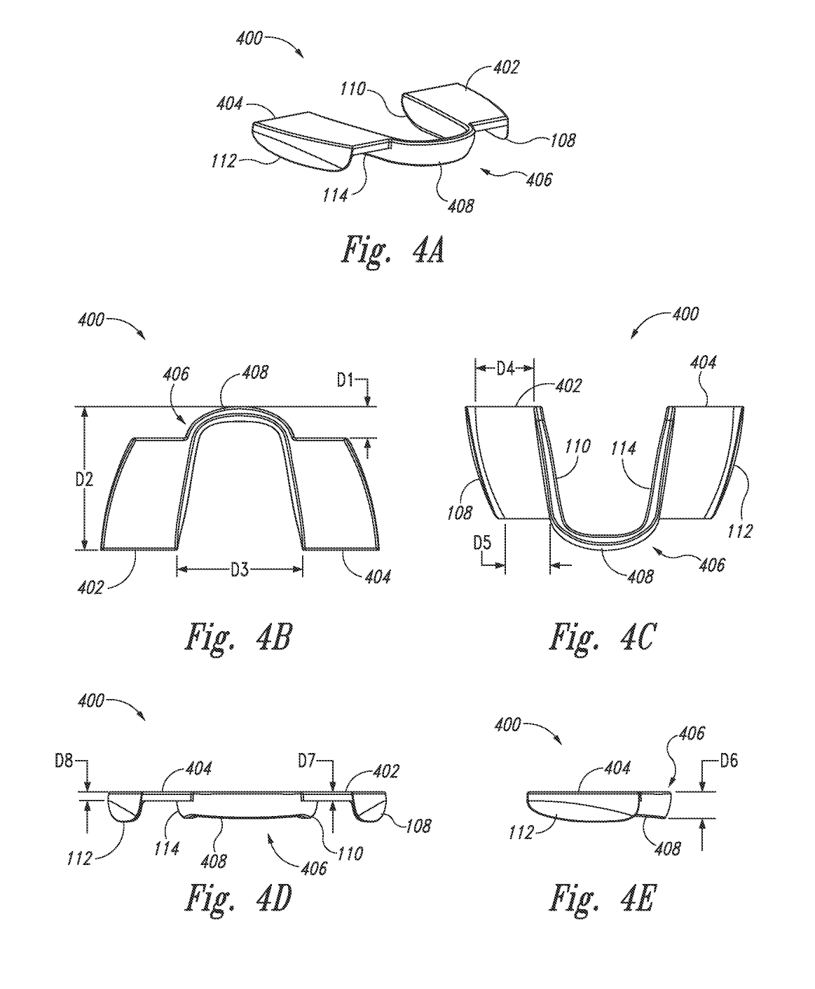

FIGS. 4A-4E are perspective, plan, inverted plan, front elevation, and side elevation views, respectively, illustrating a fourth orthosis 400 configured in accordance with an embodiment of the present technology. The fourth orthosis 400 can include pad-shaped left and right spacers 402, 404 and a curved connector 406 extending therebetween. The connector 406 can include a flexible strap 408 extending between the left and right spacers 402, 404. The strap 408 can be configured to have anterior and posterior major sides and superior and inferior edges when the fourth orthosis 400 is operably positioned within the patient's oral cavity. In some embodiments, the fourth orthosis 400 is configured to cause a different encroachment upon the patient's resting tongue position than do the first, second, and third orthoses 100, 200, 300. For example, the superior edge of the flexible strap 408 can be configured to be closer to the patient's occlusal plane than is the superior edge of the flexible strap 116 (FIGS. 1A-3E). Other types of encroachment upon the patient's resting tongue position are also possible. Alone or in combination with spacing apart the patient's posterior teeth, encroachment upon the patient's resting tongue position can be a useful form of oral biomechanical input. When a patient is not responsive to this form of oral biomechanical input, however, it can be useful to reduce or eliminate the encroachment to enhance patient comfort.

In some embodiments, the individual first, second, third, and fourth orthoses 100, 200, 300, 400 are at least generally unitary. For example, with respect to the first orthosis 100, the left spacer 102, the right spacer 104, and the connector 106 can be integrally formed. In a particular example, the first, second, third, and fourth orthoses 100, 200, 300, 400 are entirely or almost entirely made of a molded polymeric material (e.g., polyurethane). This type of construction can reduce manufacturing costs and/or reduce the possibility of breakage during use. Furthermore, the left spacers 102, 202, 302, 402 and/or the right spacers 104, 204, 304, 404 can be slightly compressible to facilitate clenching. For example, the left spacers 102, 202, 302, 402 and/or the right spacers 104, 204, 304, 404 individually can be configured to compress to a degree within a range from about 0.1 mm to about 1 mm or within another suitable range in response to a normal occlusive force (e.g., an average human occlusive force).

With reference to FIGS. 1A-4E together, the first, second, third, and fourth orthoses 100, 200, 300, 400 individually can have dimensions including a connector length (D1), an overall length (D2), an interior width (D3), a spacer posterior width (D4), a spacer anterior width (D5), a connector anterior height (D6), a left spacer height (D7), and a right spacer height (D8). In a particular embodiment, D1 is about 11 mm (e.g., within a range from about 9 mm to about 13 mm), D2 is about 36 mm (e.g., within a range from about 30 mm to about 42 mm), D3 is about 28 mm (e.g., within a range from about 23 mm to about 33 mm), D4 is about 13 mm (e.g., within a range from about 11 mm to about 15 mm), D5 is about 10 mm (e.g., within a range from about 8 mm to about 12 mm), and D6 is about 6 mm (e.g., within a range from about 5 mm to about 7 mm) in the first, second, and third orthoses 100, 200, 300; D1 is about 7 mm (e.g., within a range from about 6 mm to about 8 mm), D2 is about 32 mm (e.g., within a range from about 27 mm to about 35 mm), D3 is about 28 mm (e.g., within a range from about 23 mm to about 33 mm), D4 is about 13 mm (e.g., within a range from about 11 mm to about 15 mm), D5 is about 10 mm (e.g., within a range from about 8 mm to about 12 mm), and D6 is about 6 mm (e.g., within a range from about 5 mm to about 7 mm) in the fourth orthosis 400; D7 is about 2 mm (e.g., within a range from about 1 mm to about 3 mm), and D8 is about 5 mm (e.g., within a range from about 4 mm to about 6 mm) in the first orthosis 100; D7 is about 5 mm (e.g., within a range from about 4 mm to about 6 mm), and D8 is about 2 mm (e.g., within a range from about 1 mm to about 3 mm) in the second orthosis 100; D7 is about 5 mm (e.g., within a range from about 4 mm to about 6 mm), and D8 is about 5 mm (e.g., within a range from about 4 mm to about 6 mm) in the third orthosis 300; and D7 is about 2 mm (e.g., within a range from about 1 mm to about 3 mm), and D8 is about 2 mm (e.g., within a range from about 1 mm to about 3 mm) in the fourth orthosis 400. Other embodiments can have a variety of other suitable configurations and/or dimensions.

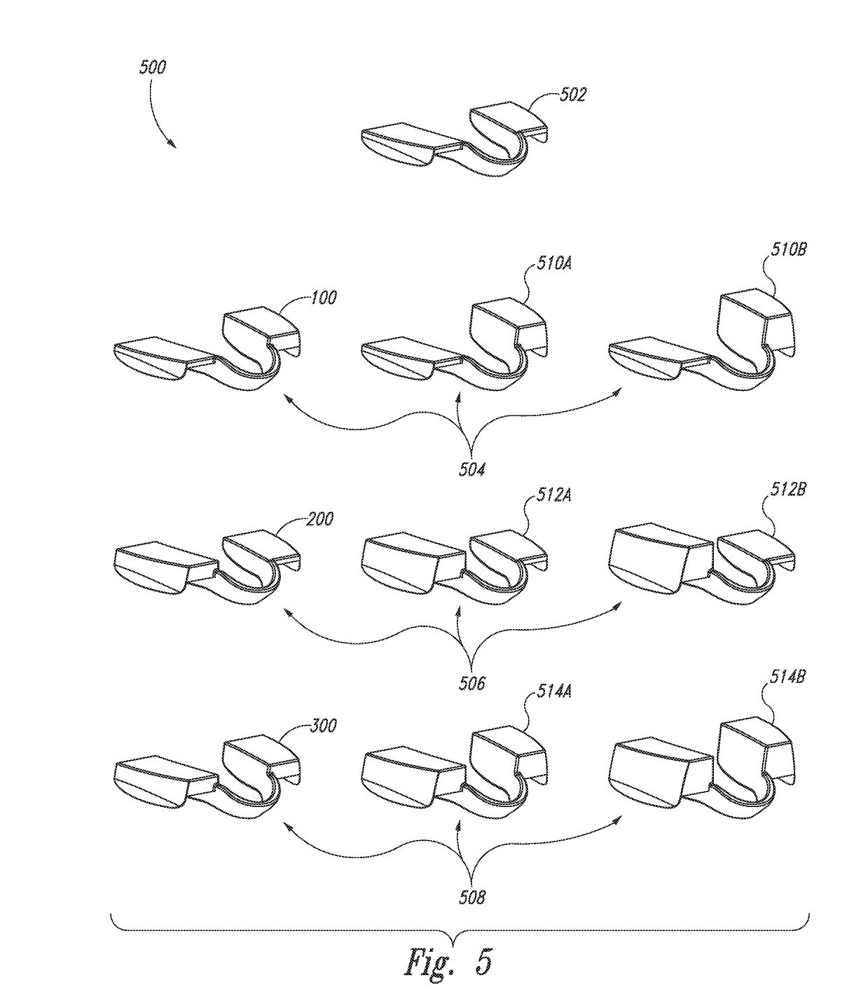

FIG. 5 is a perspective view of a screening kit 500 configured in accordance with an embodiment of the present technology. The kit 500 can include a control orthosis 502, a set of first orthoses 504, a set of second orthoses 506, and a set of third orthoses 508. The set of first orthoses 504 can include the first orthosis 100 (FIGS. 1A-1E) and two (or another suitable number) of additional first orthoses 510A, 510B. The set of second orthoses 506 can include the second orthosis 200 (FIGS. 2A-2E) and two (or another suitable number) of additional second orthoses 512A, 512B. The set of third orthoses 508 can include the third orthosis 300 (FIGS. 3A-3E) and two (or another suitable number) of additional third orthoses 514A, 514B. The first orthosis 100 and the additional first orthoses 510A, 510B within the kit 500 individually can be configured to cause different levels of greater spacing between upper left lateral posterior teeth and lower left lateral posterior teeth than between upper right lateral posterior teeth and lower right lateral posterior teeth when operably positioned within the patient's oral cavity. The second orthosis 200 and the additional second orthoses 512A, 512B within the kit 500 individually can be configured to cause different levels of greater spacing between the upper right lateral posterior teeth and the lower right lateral posterior teeth than between the upper left lateral posterior teeth and the lower left lateral posterior teeth when operably positioned within the patient's oral cavity. The third orthosis 300 and the additional third orthoses 514A, 514B within the kit 500 individually can be configured to cause different levels of at least generally symmetrical spacing between the upper and lower posterior teeth relative to the sagittal plane when operably positioned within the patient's oral cavity. In relation to the third orthosis 300 and the additional third orthoses 514A, 514B, the control orthosis 502 can be configured, for example, to cause a lower degree of at least generally symmetrical spacing between the upper and lower posterior teeth relative to the sagittal plane when operably positioned within the patient's oral cavity.

In some embodiments, the primary (e.g., the only) structural difference between members of the sets of first, second, and third orthoses 504, 506, 508, respectively, is the degree of associated left asymmetrical spacing, right asymmetrical spacing, and/or symmetrical spacing. The degrees of associated left asymmetrical spacing, right asymmetrical spacing, and/or symmetrical spacing, respectively, can vary incrementally among the members of the sets of first, second, and third orthoses 504, 506, 508, respectively, by an increment, for example, within a range from about 1 mm to about 5 mm (e.g., from about 2 mm to about 4 mm). In some embodiments, the degree of associated left asymmetrical spacing, right asymmetrical spacing, and/or symmetrical spacing, respectively, varies incrementally among the members of the sets of first, second, and third orthoses 504, 506, 508, respectively, by an increment of about 3 mm.

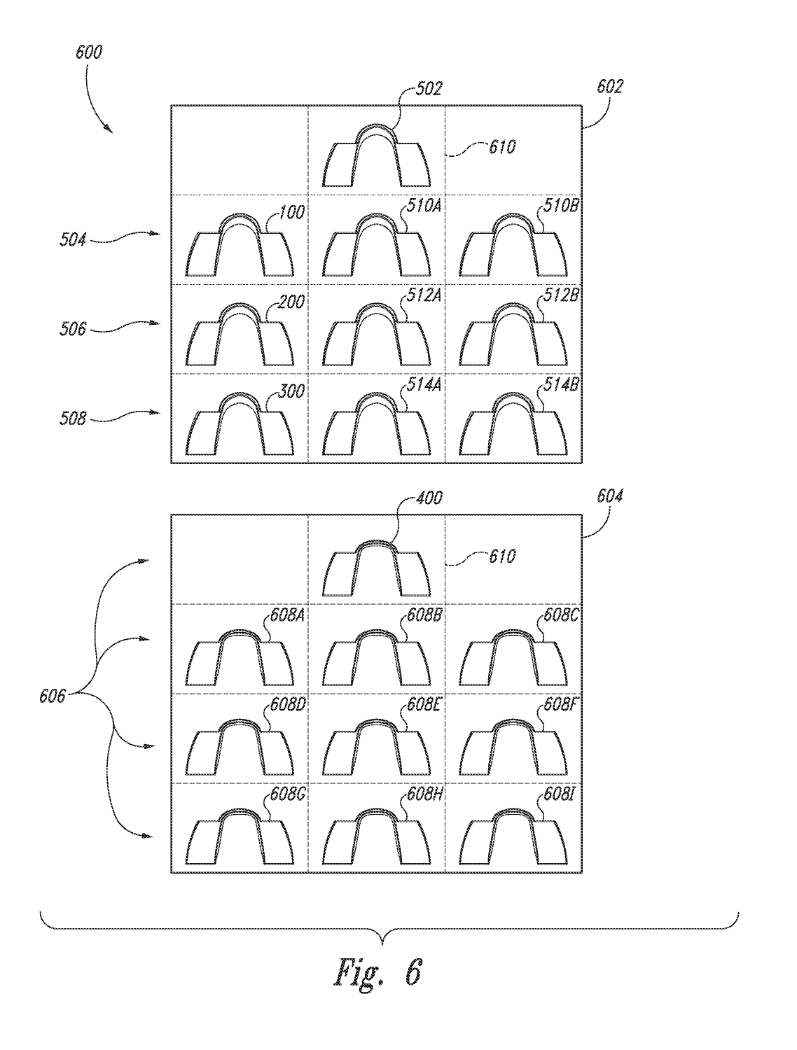

FIG. 6 is a plan view of a screening kit 600 configured in accordance with another embodiment of the present technology. The kit 600 can include a first sealed package 602 enclosing (e.g., in an at least generally sterile state) the control orthosis 502, the set of first orthoses 504, the set of second orthoses 506, and the set of third orthoses 508. The kit 600 can further include a second sealed package 604 enclosing (e.g., in an at least generally sterile state) a set of fourth orthoses 606 including the fourth orthosis 400 (FIGS. 4A-4E) and nine (or another suitable number) of additional fourth orthoses 608A-608I. In some embodiments, the first and second sealed packages 602, 604 are dividable to separate individual members of the sets of first, second, third, and fourth orthoses 504, 506, 508, 606. For example, the first and second sealed packages 602, 604 can include plastic sheets forming pouches for the individual members of the sets of first, second, third, and fourth orthoses 504, 506, 508, 606 and perforations 610 between the pouches. The fourth orthosis 400 and the additional fourth orthoses 608A-608I individually can be configured to cause one or more levels of encroachment upon the resting tongue position of the patient when operably positioned within the patient's oral cavity different than a level of encroachment individually caused by the control orthosis 502 and members of the sets of first, second, third, and fourth orthoses 504, 506, 508, 606. Furthermore, the additional fourth orthoses 608A-6081 individually can be configured to cause different levels of asymmetrical and/or symmetrical spacing between the between the patient's upper left lateral posterior teeth and the patient's lower left lateral posterior teeth than between the patient's upper right lateral posterior teeth and the patients lower right lateral posterior teeth. In some embodiments, the additional fourth orthoses 608A-6081 can be configured to cause levels of asymmetrical and/or symmetrical spacing at least partially corresponding to (e.g., matching) the levels of asymmetrical and/or symmetrical spacing caused by members of the sets of first, second, and third orthoses 504, 506, 508.

The screening kits 500, 600 and other screening kits configured in accordance with embodiments of the present technology can be used to screen biomechanical inputs for patients diagnosed with a disorder (e.g., a movement disorder, a temporomandibular joint disorder, bruxism, dysphagia, a combination thereof, or another suitable disorder). In some embodiments, all or a portion of the screening is carried out by the patient according to a set of instructions (e.g., from a clinician). One example of a screening method in accordance with an embodiment of the present technology includes operably positioning the control orthosis 502 within an oral cavity of the patient and maintaining the control orthosis 502 within the patient's oral cavity for a suitable period (e.g., a period between about 1 minute and about 48 hours, between about 1 hour and about 48 hours, or within another suitable range). Thereafter, two or more members of the set of first orthoses 504 can be sequentially operably positioned within the patient's oral cavity, each for a suitable period (e.g., a period between about 1 minute and about 48 hours, between about 1 hour and about 48 hours, or within another suitable range), two or more members of the set of second orthoses 506 can be sequentially operably positioned within the patient's oral cavity, each for a suitable period (e.g., a period between about 1 minute and about 48 hours, between about 1 hour and about 48 hours, or within another suitable range), and two or more members of the set of third orthoses 508 can be sequentially operably positioned within the patient's oral cavity, each for a suitable period (e.g., a period between about 1 minute and about 48 hours, between about 1 hour and about 48 hours, or within another suitable range). After each member of the sets of first, second, third, and fourth orthoses 504, 506, 508, 606 is tested, the patient can observe and record the effect of that member on one or more of the patient's symptoms. After screening oral biomechanical inputs using a screening method in accordance with an embodiment of the present technology, the result of the screening can be used to select the configuration of a prolonged-use orthosis (e.g., an orthosis custom made for the patient's dental anatomy). For example, the configuration of the prolonged-use orthosis can be selected based on an effectiveness (e.g., relative to the control orthosis 502) of a member of the sets of first, second, third, and fourth orthoses 504, 506, 508, 606 at reducing one or more of the patient's symptoms.

In other embodiments, the control orthosis 502, the set of first orthoses 504, the set of second orthoses 506, and the set of third orthoses 508 can be used in a different order. Furthermore, the screening method can include testing only one, two, or three among the control orthosis 502, the set of first orthoses 504, the set of second orthoses 506, and the set of third orthoses 508. For example, the method can include preliminary testing (e.g., using a screening implement as discussed above in the context of conventional screening procedures) to determine an efficacious type of oral biomechanical input (e.g., unilateral left vertical distraction, unilateral right vertical distraction, or bilateral vertical distraction) for a given patient. Thereafter, one set among the sets of first, second, and third orthoses 504, 506, 508 corresponding to the identified efficacious type of oral biomechanical input can be selected and tested to determine a degree of that types of oral biomechanical input that enhances (e.g., optimizes) the desired effect on the patient's symptom(s). Thereafter, one orthoses among the set of fourth orthoses 606 corresponding to the identified efficacious type and degree of oral biomechanical input can be selected and tested to determine whether additional benefit can be achieved by combining the efficacious type and degree of oral biomechanical input with tongue restriction. If none of the members of the sets of first, second, and third orthoses 504, 506, 508 have a beneficial effect, the fourth orthosis 400 can be tested to see if tongue restriction has a beneficial effect in isolation. Other approaches to screening patients using the screening kits 500, 600 and other screening kits configured in accordance with embodiments of the present technology are also possible.

Members of the sets of first, second, third, and fourth orthoses 504, 506, 508, 606 can be at least partially flexible to accommodate different dental anatomies. In some cases, multiple versions of the screening kits 500, 600 and other screening kits configured in accordance with embodiments of the present technology can be provided in different general sizes (e.g., small, medium, and large) and the flexibility of the members of the sets of first, second, third, and fourth orthoses 504, 506, 508, 606 can accommodate additional variation in dental anatomies among patients falling within each of the general categories.

In some cases, the anatomical effect of one or more members of the sets of first, second, third, and fourth orthoses 504, 506, 508, 606 can be visualized using a suitable imaging technique (e.g., magnetic resonance imaging). As such, members of the sets of first, second, third, and fourth orthoses 504, 506, 508, 606 can be compatible with such imaging (e.g., made of one or more materials compatible with magnetic resonance imaging). In other cases, visualizing the anatomical effect may be unnecessary. For example, the accuracy of screening methods in accordance with at least some embodiments of the present technology may allow for the identification of an efficacious type, degree, and/or combination of oral biomechanical input(s) without the need for expensive imaging procedures. This can be especially useful, for example, in developing countries and in other circumstances in which medical imaging is not widely available and/or is prohibitively expensive.

### Examples

1. An orthosis, comprising:
a left spacer configured to be clenched between upper and lower left lateral posterior teeth of a patient so as to cause a left spacing between the upper left lateral posterior teeth and the lower left lateral posterior teeth when the orthosis is operably positioned within an oral cavity of the patient;
a right spacer configured to be clenched between upper and lower right lateral posterior teeth of the patient so as to cause a right spacing between the upper right lateral posterior teeth and the lower right lateral posterior teeth when the orthosis is operably positioned within the oral cavity, the right spacing being different than the left spacing; and
a connector extending between the left and right spacers, the connector configured to accommodate anatomical differences in relative positioning of left and right lateral posterior teeth in different patients.

2. The orthosis of example 1 wherein the left and right spacers are not configured to contact anterior teeth of the patient.

3. The orthosis of example 1 wherein a difference between the left and right spacings at left and right second molars of the patient, respectively, is within a range from about 5 millimeters to about 15 millimeters.

4. The orthosis of example 1, wherein the individual left and right spacers are configured to compress to a degree within a range from about 0.1 mm to about 1 mm in response to clenching when the orthosis is operably positioned within the oral cavity.

5. The orthosis of example 1 wherein the left spacer, the right spacer, and the connector are integrally formed.

6. The orthosis of example 1 consisting essentially of molded polymeric material.

7. The orthosis of example 1, further comprising:
a left buccal flange extending downward from a buccal side of the left spacer when the orthosis is operably positioned within the oral cavity;
a left lingual flange extending downward from a lingual side of the left spacer when the orthosis is operably positioned within the oral cavity;
a right buccal flange extending downward from a buccal side of the right spacer when the orthosis is operably positioned within the oral cavity; and
a right lingual flange extending downward from a lingual side of the right spacer when the orthosis is operably positioned within the oral cavity.

8. The orthosis of example 7 wherein the connector has a left end portion directly coupled to the left lingual flange, and a right end portion directly coupled to the right lingual flange.

9. The orthosis of example 1 wherein the connector includes a flexible strap having anterior and posterior major sides and superior and inferior edges when the orthosis is operably positioned within the oral cavity.

10. The orthosis of example 9 wherein the superior edge angles downward relative to an occlusal plane of the patient as the superior edge extends mesially from the left and right spacers such that the superior edge at an anterior portion of the connector is at least about 4 millimeters below the occlusal plane when the orthosis is operably positioned within the oral cavity.

11. A screening kit, comprising:
a first orthosis configured to cause an asymmetrical spacing between upper and lower posterior teeth of a patient relative to a patient's sagittal plane when operably positioned within an oral cavity of the patient; and
a second orthosis configured to cause an asymmetrical spacing between the upper and lower posterior teeth relative to the sagittal plane when operably positioned within the oral cavity, the asymmetrical spacing caused by the second orthosis being different than the asymmetrical spacing caused by the first orthosis.

12. The screening kit of example 11 wherein the first and second orthoses are at least partially flexible to accommodate different dental anatomies.

13. The screening kit of example 11, further comprising sealed packaging enclosing the first and second orthoses, wherein the first and second orthoses are at least generally sterile within the packaging.

14. The screening kit of example 11 wherein the first and second orthoses individually are at least generally unitary.

15. The screening kit of example 11 wherein the first and second orthoses consist essentially of molded polymeric material.

16. The screening kit of example 11 wherein the first and second orthoses are disposable.

17. The screening kit of example 11 wherein:
the asymmetrical spacing caused by the first orthosis includes a first difference between left and right spacings, the left spacing being between upper and lower left lateral posterior teeth of the patient, the right spacing being between upper and lower right lateral posterior teeth of the patient;
the asymmetrical spacing caused by the second orthosis includes a second difference between the left and right spacings, the second difference being greater than the first difference; and
either the left spacing is greater than the right spacing at the asymmetrical spacings caused by both the first and second orthoses or the right spacing is greater than the left spacing at the asymmetrical spacings caused by both the first and second orthoses.

18. The screening kit of example 17, further comprising a third orthosis configured to cause an at least generally symmetrical spacing between the upper and lower posterior teeth relative to the sagittal plane when operably positioned within the oral cavity.

19. The screening kit of example 18, further comprising a fourth orthosis configured to cause a different encroachment upon a resting tongue position of the patient when operably positioned within the oral cavity than is the third orthosis.

20. The screening kit of example 11 wherein:
the asymmetrical spacing caused by the first orthosis includes greater spacing between upper left lateral posterior teeth of the patient and lower left lateral posterior teeth of the patient than between upper right lateral posterior teeth of the patient and lower right lateral posterior teeth of the patient; and
the asymmetrical spacing caused by the second orthosis includes greater spacing between the upper right lateral posterior teeth and the lower right lateral posterior teeth than between the upper left lateral posterior teeth and the lower left lateral posterior teeth.

21. The screening kit of example 20, further comprising:
a set of first orthoses individually configured to cause different levels of greater spacing between the upper left lateral posterior teeth and the lower left lateral posterior teeth than between the upper right lateral posterior teeth and the lower right lateral posterior teeth when operably positioned within the oral cavity; and
a set of second orthoses individually configured to cause different levels of greater spacing between the upper right lateral posterior teeth and the lower right lateral posterior teeth than between the upper left lateral posterior teeth and the lower left lateral posterior teeth when operably positioned within the oral cavity.

22. The screening kit of example 21, further comprising a set of third orthoses individually configured to cause different levels of at least generally symmetrical spacing between the upper and lower posterior teeth relative to the sagittal plane when operably positioned within the oral cavity.

23. The screening kit of example 22, further comprising a set of fourth orthoses individually configured to cause one or more levels of encroachment upon a resting tongue position of the patient when operably positioned within the oral cavity different than a level of encroachment caused by the individual third orthoses within the set of third orthoses, wherein the fourth orthoses within the set of fourth orthoses are individually configured to cause the different levels of greater spacing between the upper left lateral posterior teeth and the lower left lateral posterior teeth than between the upper right lateral posterior teeth and the lower right lateral posterior teeth, the different levels of greater spacing between the upper right lateral posterior teeth and the lower right lateral posterior teeth than between the upper left lateral posterior teeth and the lower left lateral posterior teeth, and the different levels of the at least generally symmetrical spacing when operably positioned within the oral cavity.

24. A method, comprising:
operably positioning a first screening orthosis within an oral cavity of a patient diagnosed with a disorder to cause an asymmetrical spacing between upper and lower posterior teeth of the patient relative to a sagittal plane, the first screening orthosis being at least partially flexible to accommodate different dental anatomies;
operably positioning a second screening orthosis within the oral cavity to cause an asymmetrical spacing between the upper and lower posterior teeth relative to the sagittal plane, the asymmetrical spacing caused by the first screening orthosis being different than the asymmetrical spacing caused by the second screening orthosis, the second screening orthosis being at least partially flexible to accommodate different dental anatomies; and
selecting a configuration of a prolonged-use orthosis for the patient based at least in part on an effectiveness of one or both of the first and second screening orthoses at reducing a symptom of the disorder, the prolonged-use orthosis being more customized for a dental anatomy of the patient than are the first and second screening orthoses.

25. The method of example 24 wherein the disorder is a movement disorder, a temporomandibular joint disorder, bruxism, dysphagia, or a combination thereof.

26. The method of example 24, further comprising:
at least generally maintaining the first screening orthosis within the oral cavity for between about 1 minute and about 48 hours after operably positioning the first screening orthosis within the oral cavity and before operably positioning the second screening orthosis within the oral cavity; and
at least generally maintaining the second screening orthosis within the oral cavity for between about 1 minute and about 48 hours after operably positioning the second screening orthosis within the oral cavity and before selecting the configuration of the prolonged-use orthosis.

27. The method of example 24 wherein:
the asymmetrical spacing caused by the first screening orthosis includes a first difference between left and right spacings, the left spacing being between upper and lower left lateral posterior teeth of the patient, the right spacing being between upper and lower right lateral posterior teeth of the patient;
the asymmetrical spacing caused by the second screening orthosis includes a second difference between the left and right spacings, the second difference being greater than the first difference; and
either the left spacing is greater than the right spacing at the asymmetrical spacings caused by both the first and second screening orthoses or the right spacing is greater than the left spacing at the asymmetrical spacings caused by both the first and second screening orthoses.

28. The method of example 24, further comprising operably positioning a third screening orthosis within the oral cavity to cause an at least generally symmetrical spacing between the upper and lower posterior teeth relative to the sagittal plane, the third screening orthosis being at least partially flexible to accommodate different dental anatomies, wherein selecting the configuration of the prolonged-use orthosis includes selecting the configuration of the prolonged-use orthosis based at least in part on an effectiveness of one or more of the first, second, and third screening orthoses at reducing the symptom, the prolonged-use orthosis being more customized for the dental anatomy of the patient than is the third screening orthosis.

29. The method of example 28, further comprising operably positioning a fourth screening orthosis within the oral cavity to cause a different encroachment upon a resting tongue position of the patient than is the third screening orthosis, the fourth screening orthosis being at least partially flexible to accommodate different dental anatomies, wherein selecting the configuration of the prolonged-use orthosis includes selecting the configuration of the prolonged-use orthosis based at least in part on an effectiveness of one or more of the first, second, third, and fourth screening orthoses at reducing the symptom, the prolonged-use orthosis being more customized for the dental anatomy of the patient than is the fourth screening orthosis.

30. The method of example 24 wherein:
the asymmetrical spacing caused by the first screening orthosis includes greater spacing between upper left lateral posterior teeth of the patient and lower left lateral posterior teeth of the patient than between upper right lateral posterior teeth of the patient and lower right lateral posterior teeth of the patient;
the asymmetrical spacing caused by the second screening orthosis includes greater spacing between the upper right lateral posterior teeth and the lower right lateral posterior teeth than between the upper left lateral posterior teeth and the lower left lateral posterior teeth;
the method further comprises—
   sequentially operably positioning two or more first screening orthoses of a set of first screening orthoses within the oral cavity, the first screening orthoses of the set of first screening orthoses being individually configured to cause different levels of greater spacing between the upper left lateral posterior teeth and the lower left lateral posterior teeth than between the upper right lateral posterior teeth and the lower right lateral posterior teeth, and
   sequentially operably positioning two or more second screening orthoses of a set of second screening orthoses within the oral cavity, the second screening orthoses of the set of second screening orthoses being individually configured to cause different levels of the greater spacing between the upper right lateral posterior teeth and the lower right lateral posterior teeth than between the upper left lateral posterior teeth and the lower left lateral posterior teeth; and
selecting the configuration of the prolonged-use orthosis includes selecting the configuration of the prolonged-use orthosis based at least in part on an effectiveness of one or more of the first screening orthoses within the set of first screening orthoses and the second screening orthoses within the set of second screening orthoses at reducing the symptom, the prolonged-use orthosis being more customized for the dental anatomy of the patient than are the first screening orthoses within the set of first screening orthoses and the second screening orthoses within the set of second screening orthoses.

31. The method of example 30, further comprising identifying a first screening orthosis or a second screening orthosis within the first and second sets of screening orthoses having the greatest effectiveness at reducing the symptom among the first and second screening orthoses within the first and second sets of screening orthoses, wherein selecting the configuration of the prolonged-use orthosis includes selecting the configuration of the prolonged-use orthosis to have a level of greater spacing between the upper left lateral posterior teeth and the lower left lateral posterior teeth than between the upper right lateral posterior teeth and the lower right lateral posterior teeth or a level of the greater spacing between the upper right lateral posterior teeth and the lower right lateral posterior teeth than between the upper left lateral posterior teeth and the lower left lateral posterior teeth at least generally corresponding to that of the identified first or second screening orthosis.

32. The method of example 30, further comprising sequentially operably positioning two or more third screening orthoses of a set of third screening orthoses within the oral cavity, the third screening orthoses of the set of third screening orthoses being individually configured to cause different levels of at least generally symmetrical spacing between the upper and lower posterior teeth relative to the sagittal plane, wherein selecting the configuration of the prolonged-use orthosis includes selecting the configuration of the prolonged-use orthosis based at least in part on an effectiveness of one or more of the first screening orthoses within the set of first screening orthoses, the second screening orthoses within the set of second screening orthoses, and the third screening orthoses within the set of third screening orthoses at reducing the symptom, the prolonged-use orthosis being more customized for the dental anatomy of the patient than are the first screening orthoses within the set of first screening orthoses, the second screening orthoses within the set of second screening orthoses, and the third screening orthoses within the set of third screening orthoses.

33. The method of example 32, further comprising:
identifying a first screening orthosis, a second screening orthosis, or a third screening orthosis within the first, second, and third sets of screening orthoses having the greatest effectiveness at reducing the symptom among the first, second, and third screening orthoses within the first, second, and third sets of screening orthoses; and
operably positioning a fourth screening orthosis within the oral cavity to cause a different encroachment upon a resting tongue position of the patient than is the identified first, second, or third screening orthosis, the fourth screening orthosis being at least partially flexible to accommodate different dental anatomies and having a level of greater spacing between the upper left lateral posterior teeth and the lower left lateral posterior teeth than between the upper right lateral posterior teeth and the lower right lateral posterior teeth or a level of the greater spacing between the upper right lateral posterior teeth and the lower right lateral posterior teeth than between the upper left lateral posterior teeth and the lower left lateral posterior teeth at least generally corresponding to that of the identified first, second, or third screening orthosis,
wherein selecting the configuration of the prolonged-use orthosis includes selecting the configuration of the prolonged-use orthosis to cause a level of encroachment upon the resting tongue position based at least in part on an effectiveness of the fourth screening orthosis relative to an effectiveness of the identified first, second, or third screening orthosis at reducing the symptom.

### CONCLUSION

This disclosure is not intended to be exhaustive or to limit the present technology to the precise forms disclosed herein. Although specific embodiments are disclosed herein for illustrative purposes, various equivalent modifications are possible without deviating from the present technology, as those of ordinary skill in the relevant art will recognize. In some cases, well-known structures and functions have not been shown or described in detail to avoid unnecessarily obscuring the description of the embodiments of the present technology. Although steps of methods may be presented herein in a particular order, alternative embodiments may perform the steps in a different order. Similarly, certain aspects of the present technology disclosed in the context of particular embodiments can be combined or eliminated in other embodiments. While advantages associated with certain embodiments of the present technology may have been disclosed in the context of those embodiments, other embodiments can also exhibit such advantages, and not all embodiments need necessarily exhibit such advantages or other advantages disclosed herein to fall within the scope of the present technology. Accordingly, this disclosure and associated technology can encompass other embodiments not expressly shown or described herein.

Throughout this disclosure, the singular terms “a,” “an,” and “the” include plural referents unless the context clearly indicates otherwise. Similarly, unless the word “or” is expressly limited to mean only a single item exclusive from the other items in reference to a list of two or more items, then the use of “or” in such a list is to be interpreted as including (a) any single item in the list, (b) all of the items in the list, or (c) any combination of the items in the list. Additionally, the terms “comprising” and the like are used throughout to mean including at least the recited feature(s) such that any greater number of the same feature and/or additional types of other features are not precluded. Directional terms, such as “upper,” “lower,” “front,” “back,” “vertical,” and “horizontal,” may be used herein to express and clarify the relationship between various elements. It should be understood that such terms do not denote absolute orientation. Reference herein to “one embodiment,” “an embodiment,” or similar formulations means that a particular feature, structure, operation, or characteristic described in connection with the embodiment can be included in at least one embodiment of the present technology. Thus, the appearances of such phrases or formulations herein are not necessarily all referring to the same embodiment. Furthermore, various particular features, structures, operations, or characteristics may be combined in any suitable manner in one or more embodiments.

I/We claim:

1. An orthosis, comprising:
a left spacer configured to be clenched between upper and lower left lateral posterior teeth of a patient so as to cause a left spacing between the upper left lateral posterior teeth and the lower left lateral posterior teeth when the orthosis is operably positioned within an oral cavity of the patient;
a right spacer configured to be clenched between upper and lower right lateral posterior teeth of the patient so as to cause a right spacing between the upper right lateral posterior teeth and the lower right lateral posterior teeth when the orthosis is operably positioned within the oral cavity, the right spacing being different than the left spacing; and
a connector extending between the left and right spacers, the connector configured to accommodate anatomical differences in relative positioning of left and right lateral posterior teeth in different patients.

2. The orthosis of claim 1 wherein the left and right spacers are not configured to contact anterior teeth of the patient.

3. The orthosis of claim 1 wherein a difference between the left and right spacings at left and right second molars of the patient, respectively, is within a range from about 5 millimeters to about 15 millimeters.

4. The orthosis of claim 1, wherein the individual left and right spacers are configured to compress to a degree within a range from about 0.1 mm to about 1 mm in response to clenching when the orthosis is operably positioned within the oral cavity.

5. The orthosis of claim 1 wherein the left spacer, the right spacer, and the connector are integrally formed.

6. The orthosis of claim 1 consisting essentially of molded polymeric material.

7. The orthosis of claim 1, further comprising:
a left buccal flange extending downward from a buccal side of the left spacer when the orthosis is operably positioned within the oral cavity;
a left lingual flange extending downward from a lingual side of the left spacer when the orthosis is operably positioned within the oral cavity;
a right buccal flange extending downward from a buccal side of the right spacer when the orthosis is operably positioned within the oral cavity; and
a right lingual flange extending downward from a lingual side of the right spacer when the orthosis is operably positioned within the oral cavity.

8. The orthosis of claim 7 wherein the connector has a left end portion directly coupled to the left lingual flange, and a right end portion directly coupled to the right lingual flange.

9. The orthosis of claim 1 wherein the connector includes a flexible strap having anterior and posterior major sides and superior and inferior edges when the orthosis is operably positioned within the oral cavity.

10. The orthosis of claim 9 wherein the superior edge angles downward relative to an occlusal plane of the patient as the superior edge extends mesially from the left and right spacers such that the superior edge at an anterior portion of the connector is at least about 4 millimeters below the occlusal plane when the orthosis is operably positioned within the oral cavity.

11. A screening kit, comprising:
a first orthosis configured to cause an asymmetrical spacing between upper and lower posterior teeth of a patient relative to a patient's sagittal plane when operably positioned within an oral cavity of the patient; and
a second orthosis configured to cause an asymmetrical spacing between the upper and lower posterior teeth relative to the sagittal plane when operably positioned within the oral cavity, the asymmetrical spacing caused by the second orthosis being different than the asymmetrical spacing caused by the first orthosis.

12. The screening kit of claim 11 wherein the first and second orthoses are at least partially flexible to accommodate different dental anatomies.

13. The screening kit of claim 11, further comprising sealed packaging enclosing the first and second orthoses, wherein the first and second orthoses are at least generally sterile within the packaging.

14. The screening kit of claim 11 wherein the first and second orthoses individually are at least generally unitary.

15. The screening kit of claim 11 wherein the first and second orthoses consist essentially of molded polymeric material.

16. The screening kit of claim 11 wherein the first and second orthoses are disposable.

17. The screening kit of claim 11 wherein:
the asymmetrical spacing caused by the first orthosis includes a first difference between left and right spacings, the left spacing being between upper and lower left lateral posterior teeth of the patient, the right spacing being between upper and lower right lateral posterior teeth of the patient;
the asymmetrical spacing caused by the second orthosis includes a second difference between the left and right spacings, the second difference being greater than the first difference; and
either the left spacing is greater than the right spacing at the asymmetrical spacings caused by both the first and second orthoses or the right spacing is greater than the left spacing at the asymmetrical spacings caused by both the first and second orthoses.

18. The screening kit of claim 17, further comprising a third orthosis configured to cause an at least generally symmetrical spacing between the upper and lower posterior teeth relative to the sagittal plane when operably positioned within the oral cavity.

19. The screening kit of claim 18, further comprising a fourth orthosis configured to cause a different encroachment upon a resting tongue position of the patient when operably positioned within the oral cavity than is the third orthosis.

20. The screening kit of claim 11 wherein:
the asymmetrical spacing caused by the first orthosis includes greater spacing between upper left lateral posterior teeth of the patient and lower left lateral posterior teeth of the patient than between upper right lateral posterior teeth of the patient and lower right lateral posterior teeth of the patient; and
the asymmetrical spacing caused by the second orthosis includes greater spacing between the upper right lateral posterior teeth and the lower right lateral posterior teeth than between the upper left lateral posterior teeth and the lower left lateral posterior teeth.

21. The screening kit of claim 20, further comprising:
a set of first orthoses individually configured to cause different levels of greater spacing between the upper left lateral posterior teeth and the lower left lateral posterior teeth than between the upper right lateral posterior teeth and the lower right lateral posterior teeth when operably positioned within the oral cavity; and
a set of second orthoses individually configured to cause different levels of greater spacing between the upper right lateral posterior teeth and the lower right lateral posterior teeth than between the upper left lateral posterior teeth and the lower left lateral posterior teeth when operably positioned within the oral cavity.

22. The screening kit of claim 21, further comprising a set of third orthoses individually configured to cause different levels of at least generally symmetrical spacing between the upper and lower posterior teeth relative to the sagittal plane when operably positioned within the oral cavity.

23. The screening kit of claim 22, further comprising a set of fourth orthoses individually configured to cause one or more levels of encroachment upon a resting tongue position of the patient when operably positioned within the oral cavity different than a level of encroachment caused by the individual third orthoses within the set of third orthoses, wherein the fourth orthoses within the set of fourth orthoses are individually configured to cause the different levels of greater spacing between the upper left lateral posterior teeth and the lower left lateral posterior teeth than between the upper right lateral posterior teeth and the lower right lateral posterior teeth, the different levels of greater spacing between the upper right lateral posterior teeth and the lower right lateral posterior teeth than between the upper left lateral posterior teeth and the lower left lateral posterior teeth, and the different levels of the at least generally symmetrical spacing when operably positioned within the oral cavity.

24. A method, comprising:
operably positioning a first screening orthosis within an oral cavity of a patient diagnosed with a disorder to cause an asymmetrical spacing between upper and lower posterior teeth of the patient relative to a sagittal plane, the first screening orthosis being at least partially flexible to accommodate different dental anatomies;
operably positioning a second screening orthosis within the oral cavity to cause an asymmetrical spacing between the upper and lower posterior teeth relative to the sagittal plane, the asymmetrical spacing caused by the first screening orthosis being different than the asymmetrical spacing caused by the second screening orthosis, the second screening orthosis being at least partially flexible to accommodate different dental anatomies; and
selecting a configuration of a prolonged-use orthosis for the patient based at least in part on an effectiveness of one or both of the first and second screening orthoses at reducing a symptom of the disorder, the prolonged-use orthosis being more customized for a dental anatomy of the patient than are the first and second screening orthoses.

25. The method of claim 24 wherein the disorder is a movement disorder, a temporomandibular joint disorder, bruxism, dysphagia, or a combination thereof.

26. The method of claim 24, further comprising:
at least generally maintaining the first screening orthosis within the oral cavity for between about 1 minute and about 48 hours after operably positioning the first screening orthosis within the oral cavity and before operably positioning the second screening orthosis within the oral cavity; and
at least generally maintaining the second screening orthosis within the oral cavity for between about 1 minute and about 48 hours after operably positioning the second screening orthosis within the oral cavity and before selecting the configuration of the prolonged-use orthosis.

27. The method of claim 24 wherein:
the asymmetrical spacing caused by the first screening orthosis includes a first difference between left and right spacings, the left spacing being between upper and lower left lateral posterior teeth of the patient, the right spacing being between upper and lower right lateral posterior teeth of the patient;
the asymmetrical spacing caused by the second screening orthosis includes a second difference between the left and right spacings, the second difference being greater than the first difference; and
either the left spacing is greater than the right spacing at the asymmetrical spacings caused by both the first and second screening orthoses or the right spacing is greater than the left spacing at the asymmetrical spacings caused by both the first and second screening orthoses.

28. The method of claim 24, further comprising operably positioning a third screening orthosis within the oral cavity to cause an at least generally symmetrical spacing between the upper and lower posterior teeth relative to the sagittal plane, the third screening orthosis being at least partially flexible to accommodate different dental anatomies, wherein selecting the configuration of the prolonged-use orthosis includes selecting the configuration of the prolonged-use orthosis based at least in part on an effectiveness of one or more of the first, second, and third screening orthoses at reducing the symptom, the prolonged-use orthosis being more customized for the dental anatomy of the patient than is the third screening orthosis.

29. The method of claim 28, further comprising operably positioning a fourth screening orthosis within the oral cavity to cause a different encroachment upon a resting tongue position of the patient than is the third screening orthosis, the fourth screening orthosis being at least partially flexible to accommodate different dental anatomies, wherein selecting the configuration of the prolonged-use orthosis includes selecting the configuration of the prolonged-use orthosis based at least in part on an effectiveness of one or more of the first, second, third, and fourth screening orthoses at reducing the symptom, the prolonged-use orthosis being more customized for the dental anatomy of the patient than is the fourth screening orthosis.

30. The method of claim 24 wherein:
the asymmetrical spacing caused by the first screening orthosis includes greater spacing between upper left lateral posterior teeth of the patient and lower left lateral posterior teeth of the patient than between upper right lateral posterior teeth of the patient and lower right lateral posterior teeth of the patient;
the asymmetrical spacing caused by the second screening orthosis includes greater spacing between the upper right lateral posterior teeth and the lower right lateral posterior teeth than between the upper left lateral posterior teeth and the lower left lateral posterior teeth;
the method further comprises—
sequentially operably positioning two or more first screening orthoses of a set of first screening orthoses within the oral cavity, the first screening orthoses of the set of first screening orthoses being individually configured to cause different levels of greater spacing between the upper left lateral posterior teeth and the lower left lateral posterior teeth than between the upper right lateral posterior teeth and the lower right lateral posterior teeth, and
sequentially operably positioning two or more second screening orthoses of a set of second screening orthoses within the oral cavity, the second screening orthoses of the set of second screening orthoses being individually configured to cause different levels of the greater spacing between the upper right lateral posterior teeth and the lower right lateral posterior teeth than between the upper left lateral posterior teeth and the lower left lateral posterior teeth; and
selecting the configuration of the prolonged-use orthosis includes selecting the configuration of the prolonged-use orthosis based at least in part on an effectiveness of one or more of the first screening orthoses within the set of first screening orthoses and the second screening orthoses within the set of second screening orthoses at reducing the symptom, the prolonged-use orthosis being more customized for the dental anatomy of the patient than are the first screening orthoses within the set of first screening orthoses and the second screening orthoses within the set of second screening orthoses.

31. The method of claim 30, further comprising identifying a first screening orthosis or a second screening orthosis within the first and second sets of screening orthoses having the greatest effectiveness at reducing the symptom among the first and second screening orthoses within the first and second sets of screening orthoses, wherein selecting the configuration of the prolonged-use orthosis includes selecting the configuration of the prolonged-use orthosis to have a level of greater spacing between the upper left lateral posterior teeth and the lower left lateral posterior teeth than between the upper right lateral posterior teeth and the lower right lateral posterior teeth or a level of the greater spacing between the upper right lateral posterior teeth and the lower right lateral posterior teeth than between the upper left lateral posterior teeth and the lower left lateral posterior teeth at least generally corresponding to that of the identified first or second screening orthosis.

32. The method of claim 30, further comprising sequentially operably positioning two or more third screening orthoses of a set of third screening orthoses within the oral cavity, the third screening orthoses of the set of third screening orthoses being individually configured to cause different levels of at least generally symmetrical spacing between the upper and lower posterior teeth relative to the sagittal plane, wherein selecting the configuration of the prolonged-use orthosis includes selecting the configuration of the prolonged-use orthosis based at least in part on an effectiveness of one or more of the first screening orthoses within the set of first screening orthoses, the second screening orthoses within the set of second screening orthoses, and the third screening orthoses within the set of third screening orthoses at reducing the symptom, the prolonged-use orthosis being more customized for the dental anatomy of the patient than are the first screening orthoses within the set of first screening orthoses, the second screening orthoses within the set of second screening orthoses, and the third screening orthoses within the set of third screening orthoses.

33. The method of claim 32, further comprising:
identifying a first screening orthosis, a second screening orthosis, or a third screening orthosis within the first, second, and third sets of screening orthoses having the greatest effectiveness at reducing the symptom among the first, second, and third screening orthoses within the first, second, and third sets of screening orthoses; and
operably positioning a fourth screening orthosis within the oral cavity to cause a different encroachment upon a resting tongue position of the patient than is the identified first, second, or third screening orthosis, the fourth screening orthosis being at least partially flexible to accommodate different dental anatomies and having a level of greater spacing between the upper left lateral posterior teeth and the lower left lateral posterior teeth than between the upper right lateral posterior teeth and the lower right lateral posterior teeth or a level of the greater spacing between the upper right lateral posterior teeth and the lower right lateral posterior teeth than between the upper left lateral posterior teeth and the lower left lateral posterior teeth at least generally corresponding to that of the identified first, second, or third screening orthosis,
wherein selecting the configuration of the prolonged-use orthosis includes selecting the configuration of the prolonged-use orthosis to cause a level of encroachment upon the resting tongue position based at least in part on an effectiveness of the fourth screening orthosis relative to an effectiveness of the identified first, second, or third screening orthosis at reducing the symptom.
